(19) United States
(12) Patent Application Publication
(10) Pub. No.: US 2013/0184550 A1
(43) Pub. Date: Jul. 18, 2013

(54) MRI-COMPATIBLE IMPLANTABLE MEDICAL LEAD

(71) Applicants: Mikael Forslund, Bromma (SE); Andreas Ornberg, Jarfalla (SE)

(72) Inventors: Mikael Forslund, Bromma (SE); Andreas Ornberg, Jarfalla (SE)

(73) Assignee: ST. JUDE MEDICAL AB, Jarfalla (SE)

(21) Appl. No.: 13/825,117

(22) PCT Filed: Sep. 19, 2011
(86) PCT No.: PCT/EP2011/066235

§ 371 Date: Mar. 19, 2013

(30) Foreign Application Priority Data
Sep. 20, 2010 (SE) PCT SE2010 051002

(51) Int. Cl. A61B 5/055; A61N 1/05; H01F 41/06; A61B 5/04

(52) U.S. Cl. CPC A61B 5/055; A61B 5/04; A61N 1/05; H01F 41/0616
USPC 600/374; 29/605

(57) ABSTRACT

An implantable medical lead (1) comprises multiple electrodes (22, 24, 26, 28) arranged at a distal end (2), multiple electrode terminals (32, 34, 36, 38) at a proximal end (3) and a lead body (4) with an insulating tubing (40). A conductor coil (10) comprises a coiled tubular insulator (19) having multiple separate lumens (12, 14, 16, 18). Each lumen (12, 14, 16, 18) houses a respective conductor (11, 13, 15, 7), which is movable in the lumen (12, 14, 16, 18) relative the coiled tubular insulator (19). The conductor coil (10) is arranged in a bore (42) of the insulating tubing (40) and each conductor (11, 13, 15, 7) is electrically connected to one of the electrodes (22, 24, 26, 28) and one of the electrode terminals (32, 34, 36, 38).

### TECHNICAL FIELD

The present invention generally relates to implantable medical leads and in particular to MRI-compatible implantable medical leads.

### BACKGROUND

Magnetic resonance imaging (MRI) is a noninvasive medical imaging technique used in radiology to visualize detailed internal structures and functions of the body of a patient. MRI generally provides much greater contrast between different soft tissues of the body than computed tomography does, making it especially useful in neurological, musculoskeletal, cardiovascular and oncological imaging.

MRI uses a powerful magnetic field to align the nuclear magnetization of, usually, hydrogen atoms in water in the body. Radio frequency (RF) fields are used to systematically alter the alignment of this magnetization. This causes the hydrogen nuclei to produce a rotating magnetic field detectable by the MRI scanner.

The electromagnetic radiation produced in the MRI may, however, be picked up by an implantable medical lead, which then acts as an antenna. The captured electromagnetic radiation will therefore induce currents in the lead, which causes heating on the stimulation and sensing electrodes of the lead. The generated heat is emitted to the surrounding tissue, such as endocardium, where it can cause injuries to the patient.

It is, though, a desire within the field to allow MRI imaging also for patients having implantable medical leads, in particular since MRI is advantageous for imaging cardiovascular tissue.

US 2009/0281608 A1 relates to medical electrical leads with spacer elements to be MRI-compatible. The medical electrical lead comprises a proximal connector, an insulated lead body including at least one electrode, a helically coiled conductor wire and a helically coiled spacer element interstitially disposed between adjacent turns of the conductor wire.

U.S. Pat. No. 7,610,101 B2 relates to a lead assembly for an implantable medical device. The lead assembly comprises a lead body having a first portion adapted for coupling to a pulse generator and a second portion adapted for implantation in or near a heart. First and second co-radial conductive coils are positioned within the lead body and electrically isolated from each other. The second conductive coil is coupled to a tip electrode located at the second portion. The first conductive coil extends past a ring electrode and transitions to a non-coiled region, which extends back to and couples to the ring electrode.

U.S. Pat. No. 6,925,334 B1 discloses an implantable lead with a lead body defining a longitudinally-extending lumen and a plurality of individual conductors contained in the lumen. The plurality of individual conductors share a common insulating coating obtained by (co-)extrusion, spraying or flood coating.

There is still a need for a design of implantable medical leads that are MRI-compatible and that can be easily manufactured without requiring several additional lead components to render the implantable medical lead MRI-compatible.

### SUMMARY

It is a general objective to provide an implantable medical lead.

It is a particular objective to provide an implantable medical lead that can be designed to be MRI-compatible and that is easy to manufacture.

These and other objectives are met by embodiments as disclosed herein.

Briefly an implantable medical lead comprises multiple electrodes arranged in connection with a distal end of the implantable medical lead. An opposite, proximal end of the implantable medical lead is configured to be mechanically and electrically connected to an implantable medical device. Multiple electrode terminals are arranged in connection with this proximal end. A lead body comprising an insulating tubing having a bore is running from the proximal end to the distal end. In the bore, a conductor coil is arranged. This conductor coil comprises a coiled tubular insulator having multiple separate and electrically isolated lumens. Each of the multiple lumens furthermore houses a conductor which is electrically connected to an electrode at the distal end and an electrode terminal at the proximal end. The lumens of the coiled tubular insulator are designed so that their cross-sectional dimensions relative the cross-sectional dimensions of the conductors enable each conductor to be movable in its lumen relative the coiled tubular insulator.

As a consequence, the conductor coil will be handled as a single unit with the coiled tubular insulator and the conductors present in different lumens formed in the tubular insulator. This significantly facilitates assembly of the implantable medical lead. Furthermore, the design of the lumen cross-sectional dimensions relative the cross-section dimensions of the conductors enables the conductors to be easily introduced in the lumens during manufacture. This enables the coiled tubular insulator to be manufactured separately from the conductors.

Additionally, the arrangement of the conductors inside the lumens of the coiled tubular insulator enables the implantable medical lead to be MRI-compatible by providing increased lead inductance and capacitance, which in turn reduce any tissue heating induced by an applied RF field during MRI scanning.

An aspect also relates to a method of manufacturing an implantable medical lead. In an embodiment of the method a polymer is extruded to form a tubular insulator having multiple separate lumens. A respective conductor is introduced in each of the lumens. The cross-sectional dimensions of the lumens are selected relative the cross-sectional dimensions of the conductors to enable movement of each conductor in its lumen and relative the tubular insulator. The tubular insulator is furthermore coiled, prior or preferably after introducing the conductors in the lumens, to form a conductor coil comprising the coiled tubular insulator. The method further involves introducing the conductor coil in a bore of an insulating tubing of a lead body. Each conductor is then electrically connected to an electrode arranged in connection with the distal end of the implantable medical device and an electrode terminal arranged in connection with the proximal end of the implantable medical device.

Another aspect relates to a method of manufacturing an implantable medical lead. An insulating core structure having a cross-shaped cross section is provided. The particular cross sectional shape of the core structure defines four open channels. A respective conductor is then arranged in each of the four open channels. The insulating core structure with the conductors is introduced into a bore of an insulating tubing to form a tubular insulator having four separate lumens with a respective conductor in each lumen. Alternatively, the insulating core structure is introduced into the bore of the first insulating tubing before introducing the conductors in the formed lumens. The cross-sectional dimensions of the lumens are selected relative the cross-sectional dimensions of the conductors to enable movement of each conductor in its lumen and relative the tubular insulator. The formed tubular insulator is coiled to get the conductor coil. The method further involves introducing the conductor coil in a bore of an insulating tubing of a lead body. Each conductor is then electrically connected to an electrode arranged in connection with the distal end of the implantable medical device and an electrode terminal arranged in connection with the proximal end of the implantable medical device.

### BRIEF DESCRIPTION OF THE DRAWINGS

The invention, together with further objects and advantages thereof, may best be understood by making reference to the following description taken together with the accompanying drawings, in which:

FIG. 1 is an illustration of an implantable medical lead according to an embodiment;

FIG. 2 is an illustration of a coaxial conductor coil to be used in an implantable medical lead according to an embodiment;

FIG. 3 is an illustration of a coaxial conductor coil introduced into a bore of an insulating tubing shown in cross-section of an implantable medical lead according to an embodiment;

FIG. 4 is an illustration of a coaxial conductor coil introduced into a bore of an insulating tubing of an implantable medical lead according to an embodiment;

FIG. 5 is an illustration of a coaxial conductor coil introduced into a bore of an insulating tubing of an implantable medical lead according to another embodiment;

FIG. 6 is a flow diagram illustrating a method of manufacturing an implantable medical lead according to an embodiment.

FIG. 7 is a flow diagram illustrating a method of manufacturing an implantable medical lead according to another embodiment; and

FIGS. 8A and 8B schematically illustrate the manufacture of a tubular insulator according to an embodiment.

### DETAILED DESCRIPTION

Throughout the drawings, the same reference numbers are used for similar or corresponding elements.

An aspect of the embodiments relates to an implantable medical lead and in particular such an implantable medical lead that is suitable for implantation in an animal subject, preferably mammalian subject and more preferably a human subject. The implantable medical lead can additionally be used in subjects exposed to an MRI system or scanner and is therefore MRI-compatible.

MRI-compatible as used herein implies that any heating of electrodes in connection with the distal end of the implantable medical lead caused by a current induced by RF fields of the MRI system is at an acceptable level to thereby not cause or at least reduce the risk of causing significant injuries to surrounding tissue in the subject body or damage to internal lead parts.

Although the implantable medical lead of the embodiments can be designed to be MRI-compatible it can also be used for subjects that will never be exposed to any MRI system. Hence, the implantable medical lead will also have significant advantages, in particular during the manufacture of the implantable medical lead, as compared to prior art solutions.

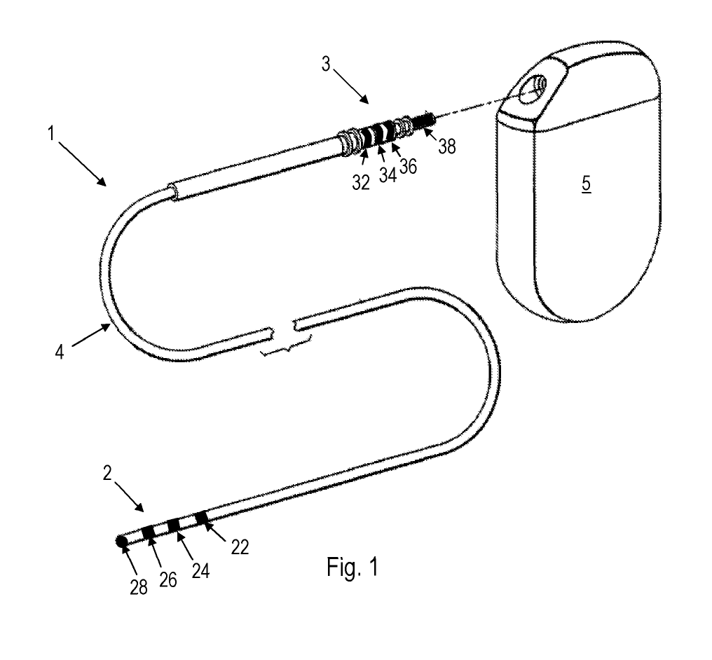

FIG. 1 is a schematic overview of an implantable medical lead 1 according to an embodiment. The implantable medical lead 1 comprises a distal end 2 designed to be introduced into a suitable pacing site to enable delivery of pacing pulses and sensing electric activity of the tissue, such as heart, at the particular pacing site. Multiple electrodes 22-28, generally denoted pacing and sensing electrodes in the art, are arranged in connection with the distal end 2. It is these electrodes 22-28 that deliver pacing pulses to the tissue and captures electric signals originating from the tissue. The implantable medical lead 1 comprises multiple, i.e. at least two, electrodes 22-28 in connection with the distal end 2. In FIG. 1, a so-called quadropolar implantable medical lead 1 has been illustrated having four electrodes 22-28. This should merely be seen as an illustrative example and the implantable medical lead 1 could instead be a bipolar lead with two electrodes, a tripolar lead with three electrodes or indeed have five or more electrodes.

An opposite or proximal end 3 of the implantable medical lead 1 is configured to be mechanically and electrically connected to an implantable medical device (IMD) 5. The IMD 5 can be any implantable medical device used in the art for generating and applying, through the implantable medical lead 1, electric pulses or shocks to tissues. The IMD 5 is advantageously a pacemaker, defibrillator or cardioverter to thereby have the implantable medical lead 1 implanted in or in connection to a ventricle or atrium of the heart. However, also other types of IMDs 5 that are not designed for cardiac applications, such as neurological stimulator, physical signal recorders, etc. can be used as IMDs 5 to which the implantable medical lead 1 can be connected.

The proximal end 3 comprises multiple electrode terminals 32-38 that provide the electric interface of the implantable medical lead 1 towards the IMD 5. Thus, each electrode terminal 32-38 is connected to a respective connector terminal in the IMD 5 to thereby provide electric connection between the IMD 5 and the electrodes 22-28 through the electrode terminals 32-38 and a conductor coil, to be further described herein.

The implantable medical lead 1 typically comprises a respective electrode terminal 32-38 for each electrode 22-28 in connection with the distal end 2.

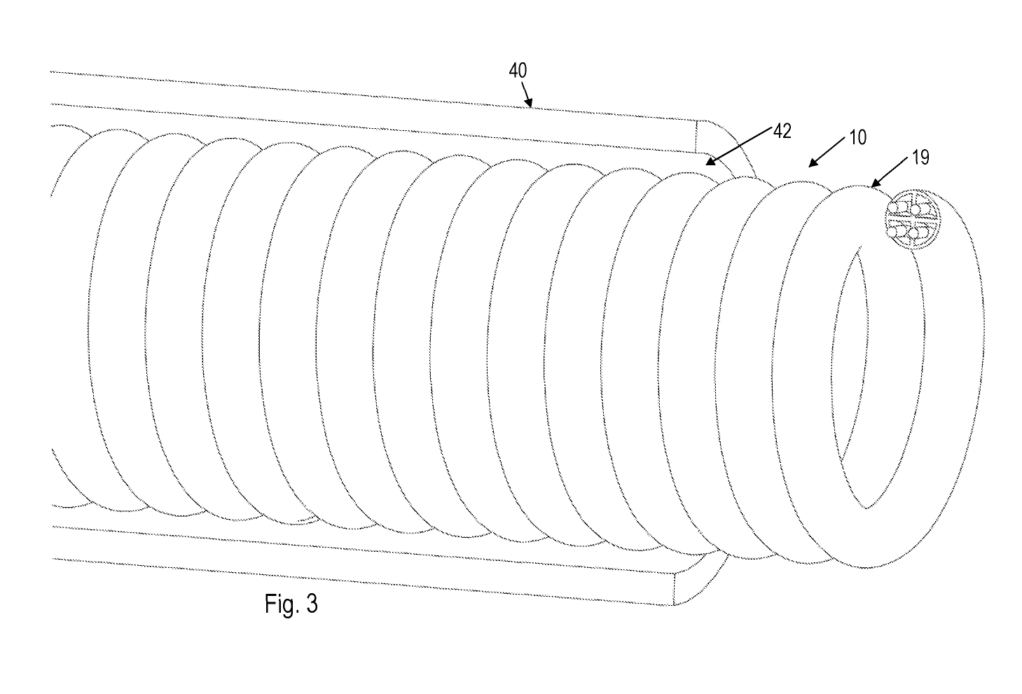

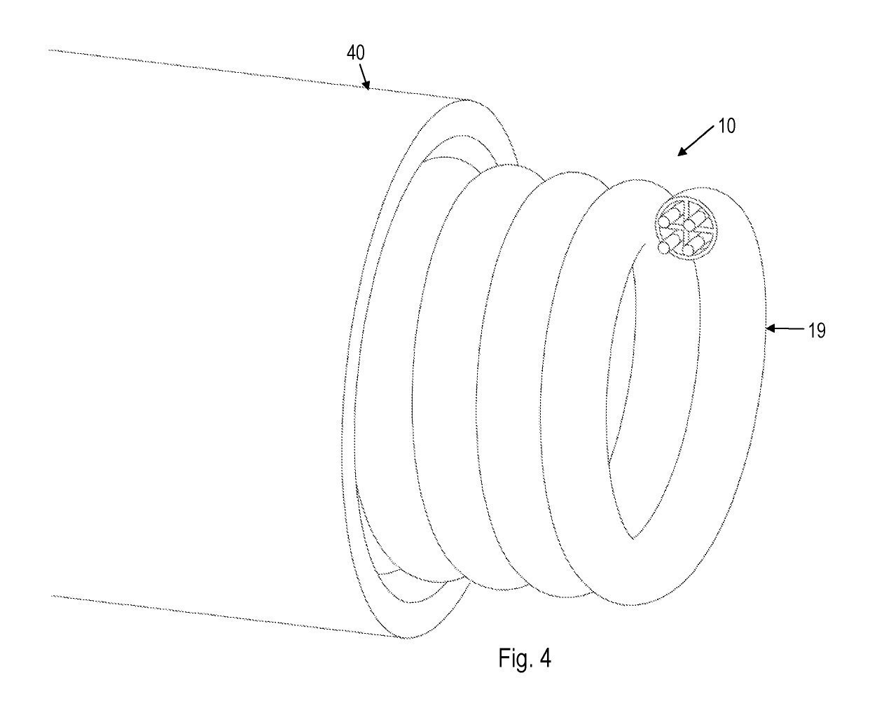

The implantable medical lead 1 also comprises a lead body 4 running from the proximal end 3 to the distal end 2. This lead body 4 comprises an insulating tubing 40 having a bore 42 (see FIGS. 3-5). This bore 42 is designed and dimensioned to house a conductor coil 10 that provides the electrical connection between the multiple electrodes 22-28 and the multiple electrode terminals 32-38.

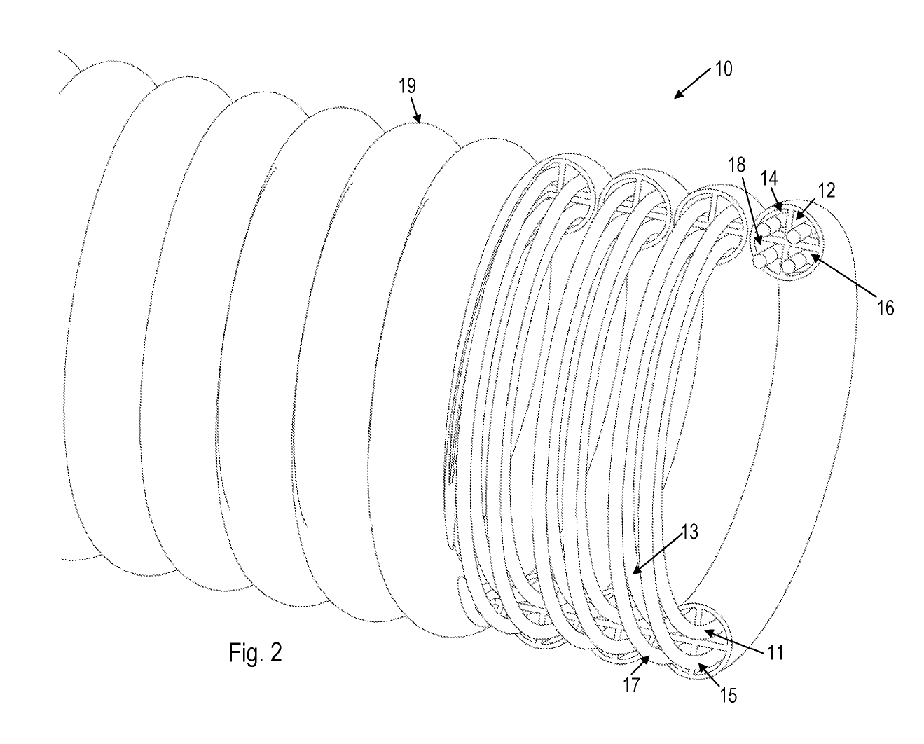

FIG. 2 illustrates this conductor coil 10 in more detail according to an embodiment. The conductor coil 10 comprises a coiled tubular insulator 19 having multiple separate lumens 12, 14, 16, 18. The outer diameter of the conductor coil 10 is selected to not exceed the inner diameter of the bore 42 of the insulating tubing 40, see FIGS. 3-5. Hence the conductor coil 10 can easily be arranged inside the bore 42.

The lumens 12, 14, 16, 18 run like channels in the coiled tubular insulator 19 and preferably as multiple parallel channels. Each lumen 12, 14, 16, 18 houses a conductor 11, 13, 15, 17 that runs in the lumen 12, 14, 16, 18. Each conductor 11, 13, 15, 17 is furthermore electrically connected to an electrode in connection with the distal end of the implantable medical lead and to an electrode terminal in connection with the proximal end of the implantable medical lead. Thus, the conductors 11, 13, 15, 17 in the lumens 12, 14, 16, 18 of the coiled tubular insulator 19 provide the electric connection between the electrodes and the electrode terminals.

According to the embodiments the dimensions of the lumens 12, 14, 16, 18 and more particularly the cross-sectional dimensions of the lumens 12, 14, 16, 18 are selected and defined relative the cross-sectional dimensions, typically diameters, of the conductors 11, 13, 15, 17. This means that the conductors 11, 13, 15, 17 are movable in the lumens 12, 14, 16, 18 relative the coiled tubular insulator 19. Thus, the cross-sectional dimensions of the lumens 12, 14, 16, 18 are defined relative the cross-sectional dimensions of the conductors 11, 13, 15, 17 to enable movement of each respective conductor 11, 13, 15, 17 in its lumen 12, 14, 16, 18 and relative the coiled tubular insulator 19.

In a particular embodiment, the largest outer cross-sectional dimension of the conductors 11, 13, 15, 17, 5 typically the outer diameter of the conductors 11, 13, 15, 17, is smaller than a smallest inner cross-sectional dimension of the lumens 12, 14, 16, 18, such as inner diameter of the lumens 12, 14, 16, 18. These relative sizes of the cross-sectional dimensions of the lumens 12, 14, 16, 18 and the conductors 11, 13, 15, 17 are clearly evident from FIGS. 2-5.

This particular embodiment of defining the respective cross-sectional dimensions enables an easy manufacture of the implantable medical lead which is further disclosed herein. Thus, only with such relative dimension sizes that enables movement of the conductors 11, 13, 15, 17 in the lumens 12, 14, 16, 18 can the implantable medical lead be easily but also safely produced.

In a particular embodiment, each conductor 11, 13, 15, 17 is connected to a respective electrode and a respective electrode terminal. Thus, in such a case there is a one-to-one relationship between the electrodes, the electrode terminals and the conductors 11, 13, 15, 17, and also to the lumens 12, 14, 16, 18 in the coiled tubular insulator 19. This is generally preferred.

However, in some applications it could be preferred to have at least two of the conductors 11, 13, 15, 17 connected to the same electrode and the same electrode terminal. In such an application the implantable medical lead provides redundancy with regard to the number of electric conductors interconnecting at least one electrode-terminal pair.

The coiled tubular insulator 19 of the conductor coil 10 can, as has been described above, comprise two or more lumens 12, 14, 16, 18 that are electrically isolated from each other. If the coiled tubular insulator 19 comprises two such lumens, they can be arranged in various embodiments following coiling. In a first embodiment, the two lumens and the conductors therein could be coaxially arranged. This would correspond to lumens 14, 18 and conductors 13, 17 in FIG. 2 or lumens 12, 16 and conductors 11, 15 in FIG. 2. Thus, the two conductors are coaxially arranged with regard to the longitudinal axis of the conductor coil 10 and the longitudinal axis of the lead body. In such a case, the radius to the outer lumen 12, 14 from the central longitudinal axis is larger than the radius to the inner lumen 16, 18 from the central longitudinal axis. In a second embodiment, the two lumens and conductors are instead co-radially arranged. This would correspond to lumens 12, 14 and conductors 15, 17 in FIG. 2 or lumens 16, 18 and conductors 11, 13 in FIG. 2. Thus, the radiuses from the central longitudinal axis out to either of the two lumens are substantially the same.

In a particular embodiment, the coiled tubular insulator 19 has four separate lumens 12, 14, 16, 18 and 5 thereby four conductors 11, 13, 15, 17 as illustrated in FIG. 2. In such a case, the cross-sectional structure of the coiled tubular insulator 19 will define four quadrants, with a respective lumen 12, 14, 16, 18 and conductor 11, 13, 15, 17 in each quadrant.

With such a conductor coil 10 the implantable medical lead 1 typically comprises four electrodes 22-28 10 and four electrode terminals 32-38 as illustrated in FIG. 1 so that each conductor 11, 13, 15, 17 interconnects a respective electrode-terminal pair.

It is, though, possible to use the conductor coil embodiment of FIG. 2 in a bipolar implantable medical lead. In such a case, two of the conductors are electrically connected to a first electrode in connection 15 with the distal end of the implantable medical lead and a first electrode terminal in connection with the proximal end of the implantable medical lead. The remaining two conductors are electrically connected to a second electrode and a second electrode terminal.

The coiled tubular insulator 19 advantageously comprises multiple pairs or sets of co-radial lumens. In FIGS. 2 and 4-5, lumens 12, 14 form a first such pair with lumens 16, 18 constituting another pair. In such a case, the conductor coil 10 will be a co-radial, coaxial conductor coil 10 since the inner pair of co-radial lumens 16, 18 and conductors 11, 13 will be coaxial relative the outer pair of co-radial lumens 12, 14 and conductors 15, 17.

In the case of six lumens and conductors, an inner set of three co-radial lumens and conductors can be coaxially provided relative an outer set of three co-radial lumens and conductors. In an alternative approach, an inner pair of co-radial lumens and conductors is coaxially arranged relative a middle pair of co-radial lumens and conductors and an outer pair of co-radial lumens and conductors. This concept can be extended even further to eight or more lumens or conductors. However, increasing the number of lumens and conductors beyond four will generally increase both the diameter of the tubular insulator 19 and the diameter of the whole conductor coil 10. In such a case, the total thickness or diameter of the implantable medical lead could be rather large, which is generally not desirable.

The conductor coil design of the embodiments with a coiled tubular insulator having multiple electrically separated lumens with a respective conductor in each lumen provides advantages to the art of implantable medical leads. Firstly, the inclusion of the multiple lumens 12, 14, 16, 18 and the conductors 11, 13, 15, 17 in the coiled tubular insulator 19 implies that the coiled tubular insulator 19 and the conductors 11, 13, 15, 17 can be handled, during assembly of the implantable medical lead 1, as a single unit. This significantly improves the handling and speeds up the assembly process as compared to the case where multiple individual conductors need to be introduced into the bore 42 of the insulating tubing 40.

Additionally, by having the conductors 11, 13, 15, 17 present in the same electrically insulating structure, i.e. in the lumens 12, 14, 16, 18 of the coiled tubular insulator 19, the conductors 11, 13, 15, 17 can be kept at a very close distance from each other and still be electrically insulated from each other. The tight packing of the conductors 11, 13, 15, 17 and the small distance between the conductors 11, 13, 15, 17 imply that the inductance and capacitance of the conductor coil 10 are increased as compared to the coaxial conductor coils traditionally used in implantable medical leads. The increase in inductance is achieved due to the fact that the outer diameter of the conductor coil 10 can be made as large as the inner diameter of the insulating tubing 40, i.e. typically larger than for traditional implantable medical leads. In addition, or alternatively, the conductors 11, 13, 15, 17 in the lumens 12, 14, 16, 18 can be made thin to thereby have small diameters since the conductors 11, 13, 15, 17 do not need to provide any structural integrity or stability to the implantable medical lead 1 or the conductor coil 10. In clear contrast, the structural stability of the conductor coil 10 is mainly maintained by the tubular insulator 19. The conductor coil 10 can therefore be manufactured with really thin conductors 11, 13, 15, 17, such as having a diameter smaller than 0.15 mm and in particular smaller than 0.1 mm. It is in fact possible to have even thinner conductors 11, 13, 15, 17 with a diameter of no more than 0.05 mm.

The increased capacitance of the conductor coil 10 is obtained due to the reduced distance between the conductors 11, 13, 15, 17 in the conductor coil 10 as discussed above.

The high inductance and capacitance will significantly reduce any heating at the distal electrodes 22-28 in connection with an MRI scanning session.

Furthermore, by designing the tubular insulator 19 with lumen dimensions that enables the conductors 11, 13, 15, 17 to be movable inside the lumens 12, 14, 16, 18 and relative the tubular insulator 19 the implantable medical lead can be easily manufactured in few process steps but still providing sufficient high manufacturing reliability and safety. This is discussed further herein in connection with manufacturing embodiments.

The conductors 11, 13, 15, 17 can be in the form of wires, cables or coils of electrically conductive material and dimensioned to be introduced in the lumens 12, 14, 16, 18. In order to increase the inductance of the conductor coil 10 even further the conductors 11, 13, 15, 17 can be in the form of coiled wires. Hence, in such a case each lumen 12, 14, 16, 18 houses a coiled wire as conductor 11, 13, 15, 17 and the conductors 11, 13, 15, 17 in the lumens 12, 14, 16, 18 of the tubular insulator 19 are then coiled to form the final conductor coil 10.

In the case of coiled wires as conductors 11, 13, 15, 17, the cross-sectional dimensions of the lumens 12, 14, 16, 18 are defined to be sufficient large to enable movement of the coiled wires in the lumens 12, 14, 16, 18 as previously disclosed herein.

The wires or cables may additionally be surrounded by a separate insulating tubing. In such a case, each lumen comprises a respective conductor having a surrounding insulating tubing. This is, though generally not necessary from an insulation point of view but could simplify introduction of the conductors in the lumens by achieving a lower friction between the material of the coiled tubular insulator and the separate conductor insulating tubing as compared to between the conductors and the coiled tubular insulator.

The coiled tubular insulator 19 can be manufactured in various insulating materials that can be formed in the form of a tube having the multiple electrically separated lumens 12, 14, 16, 18. The coiled tubular insulator 19 will typically not come into contact with the subject body even after implantation. Hence, it is not an absolute requisite that the insulating material of the coiled tubular insulator 19 is biocompatible. However, it is generally preferred to select the insulating material from biocompatible, non-toxic materials. Non-limiting examples include silicone, polyurethane, co-polymers of polyurethane and silicone, such as Optim™, polyether ether ketone (PEEK), ultra high molecular weight polyethylene (UHMPWE or sometimes shortened to UHMW), polyether block amide (PEBA) (also known under the tradename PEBAX), polyamide or polyimide, polybuthene and polypropylene.

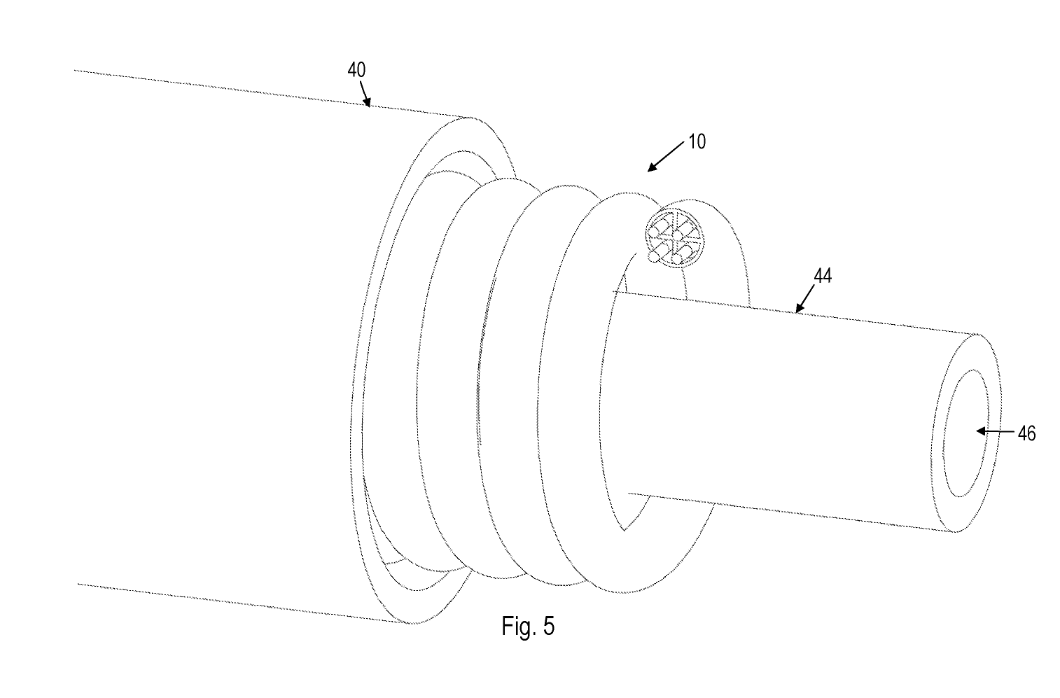

FIGS. 3-5 illustrate the conductor coil 10 when it has been introduced in the bore 42 of the insulating tubing 40 of the lead body 4. As shown in FIG. 5, in order to even further increase the stability and stiffness of the implantable medical lead, an inner insulating tubing 44 can be coaxially arranged relative the outer insulating tubing 40 and the conductor coil 10 in the lumen or channel formed by the conductor coil 10. This inner insulating tubing 44 in turn comprises a central bore 46 through which a guide wire can be introduced during implantation of the implantable medical lead, which is well known in the art. If the implantable medical lead is of a so-called active fixation type it has a fixation helix or screw that is employed to attach the implantable medical lead to a tissue. In such a case, the fixation helix is connected or attached to a screw coil or structure that can run from the proximal end of the implantable medical lead to the fixation helix and in the bore 46 of the inner insulating tubing 44. In a particular embodiment, such screw coil or structure can then be made of non-conducting material since the electrical conduction is instead performed by the conductors of the conductor coil 10.

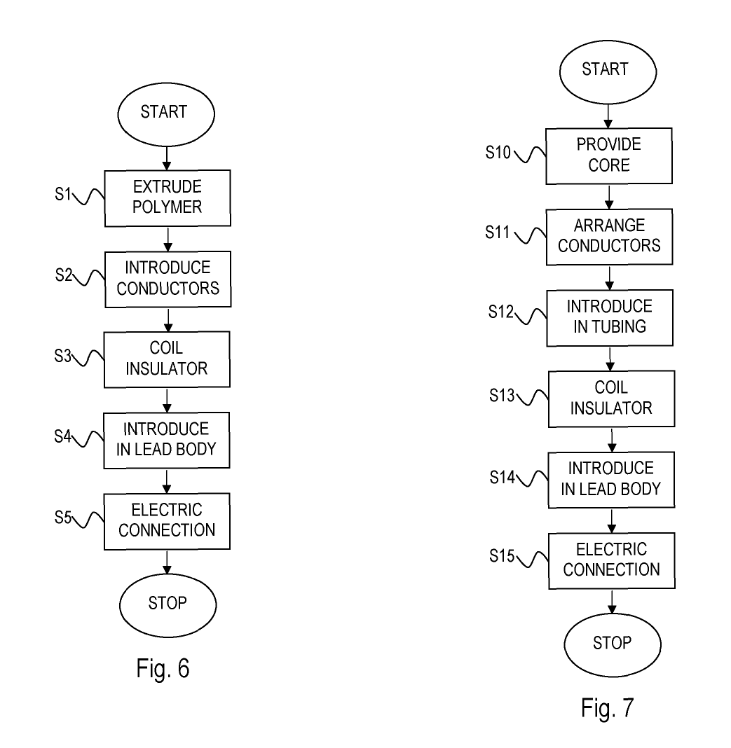

FIG. 6 is a flow diagram illustrating an embodiment of manufacturing an implantable medical lead according to an embodiment. The method starts in step S1, where a polymer is extruded to form a tubular insulator having multiple separate lumens. Extruding such multi-lumen polymers can be conducted according to techniques well known in the art. The polymer is preferably polyurethane, a co-polymer of polyurethane and silicone, such as Optim™, PEEK, UHMWPE, PEBA, polyamide or polyimide. Also thermoplastic silicone could be used. The tubular insulator is formed with the multiple lumens that have cross-sectional dimensions that are selected to be large enough to house a respective conductor and still enable the conductor to be moved inside the lumen and relative the tubular insulator. A next step S2 introduces the conductors into the respective lumens of the tubular insulator formed in step S1. This conductor introduction can be performed by pushing the conductors into the lumens. However, it is generally preferred to pull them into lumens by means of some thin wire or structure. The selection of the cross-sectional dimensions of the lumens relative the cross-sectional dimensions of the conductors simplifies the introduction of the conductors in the lumens.

The tubular insulator is further coiled to form the conductor coil having the coiled tubular insulator with the multiple lumens and conductors. This coiling is preferably performed after introducing the conductors, which has been illustrated in FIG. 6 as step S3. It could be possible to perform the coiling of the tubular insulator before introduction of the conductors, though this generally makes the introduction of the conductors much harder. The coiling of step S3 is preferably performed during heat treatment to thereby form the coiled tubular insulator once it has cooled. Following the heat treatment the coiled tubular insulator should thereby keep its coiled structure and form.

In a next step S4 the conductor coil formed in step S3 is introduced into a bore of the insulating tubing of the lead body. The conductors in the conductor coil are then, in step S5, electrically connected to the electrodes arranged in connection with the distal end of the implantable medical lead and to the electrode terminals in connection with the proximal end of the implantable medical lead as previously disclosed herein.

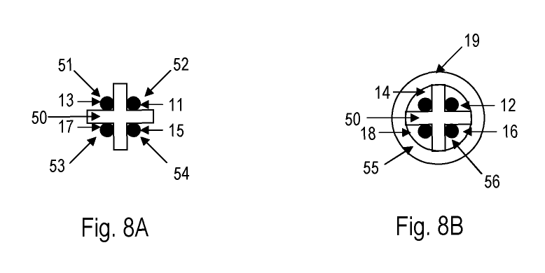

FIG. 7 is a flow diagram illustrating another embodiment of manufacturing the implantable medical lead. 5 Reference is also made to FIGS. 8A and 8B illustrating the manufacture of the tubular insulator. The method starts in step S10, where an insulating core structure 50 having a cross-shaped cross section is provided. This cross-shape implies that the insulating core structure 50 defines four open channels 51, 52, 53, 54, one in each quadrant.

A respective conductor 11, 13, 15, 17 is arranged in each of the four open channels 51, 52, 53, 54 in step S11. The insulating core structure 50 with the conductors 11, 13, 15, 17 is then introduced into a bore 56 of a first insulating tubing 55 in step S12. In an alternative approach, the insulating core structure 50 is first introduced into the bore 56 of the first insulating tubing 55 before the conductors 11, 13, 15, 17 are arranged in the four open channels 51, 52, 53, 54. Thus, in this embodiment, a respective conductor 11, 13, 15, 17 is arranged in each of the four lumens 12, 14, 16, 18 after introducing the insulating core structure 50 in the bore 56 of the first insulating tubing 55. Thus, step S12 is performed prior to step S11.

This first insulating tubing 55 is made of an elastic, deformable material to provide a tight connection between its inner bore wall and the arms of the insulating core structure 50 when the insulating core structure 50 has been introduced in the bore 56 as shown in FIG. 8B. Hence, the diameter of the bore 56 is preferably selected to be smaller than the length of two opposite arms of the insulating core structure 50 to achieve this tight connection between insulating core structure 50 and the first insulating tubing 55. The tubular insulator is then formed in this step S12 to form and enclose four lumens with a respective conductor 11, 13, 15, 17 in each lumen. Hence, the lumens are formed by the space enclosed by the first insulating tubing 55 and the insulating core structure 50. According to the embodiments, the cross-sectional dimensions of the lumens are defined relative the cross-sectional dimensions of the conductors 11, 13, 15, 17 to enable movement of the conductors 11, 13, 15, 17 in the lumens and relative the tubular insulator. The tight connection between the arms of the insulating core structure 50 and the inner wall of the first insulating tubing effectively prevents any conductor 11, 13, 15, 17 from leaving its enclosed lumen.

The first insulating tubing 55 is preferably selected among elastic and deformable elastomers and material. Non-limiting examples include silicone and a co-polymer of polyurethane and silicone, such as Optim™. The insulating core structure 50 is preferably selected among silicone, a co-polymer of polyurethane and silicone, such as Optim™, polyethylene, polybuthene, polypropylene, thermoplastic polyurethane, such as sold under tradename PELLETHANE. In a particular embodiment, the insulating core structure 50 and the first insulating tubing 55 are made of the same material.

The tubular insulator is then coiled in step S13 to form the conductor coil, which is introduced into the bore of a second insulating tubing in step S14 and the conductors are electrically connected to the electrodes and electrode terminals in step S15. These steps S13 to S15 are performed in the same way as steps S3-S5 described in connection with FIG. 6 above and are therefore not described in more detail herein.

The manufacturing method of the embodiments has several important advantages over the state of the art. For instance, U.S. Pat. No. 6,925,334 B1 cited in the background section uses an electrical conductor assembly in which multiple conductors share a common insulating coating that is thought to electrically isolate the conductors from each other. This insulating coating is applied over the conductors using extrusion, spraying or dipping techniques. However, manufacturing such an electrical conductor assembly according to the prior art is hard while simultaneously guaranteeing that the conductors are safely kept isolated from each other. The conductors used in implantable medical leads are very thin structures. Arranging such thin conductors while extruding the isolating material around the conductors while trying to keep the conductors from being pushed against each other at any point along the length of the electrical conductor assembly is difficult. Hence, with the prior art manufacturing techniques it is very cumbersome to manufacture an electrical conductor assembly that will securely isolate the conductors from each other and thereby prevent any risk of short circuit during usage of the implantable medical lead.

In clear contrast, according to the invention the tubular insulator is manufactured separately by extrusion of the polymer to form the separate lumens or by separately manufacturing the insulating core structure and the first insulating tubing. No conductors need to be kept in tight positions and separated from each other during these process steps. This further implies that the thickness of the insulating material around the conductors when introduced in the lumens of the tubular insulator can be kept within controlled margins. In the prior art there is a risk that the thickness of the isolating material between the conductors will vary along the assembly length due to the problems of keeping the conductors separated from each other at a defined distance along the whole length.

The risk of any short circuit during operation of the implantable medical lead is next to zero since the tubular insulator can be manufactured in a controlled manner with sufficient insulating material between the lumens. The lumens will therefore be kept insulated and separated from each other along the whole length of the tubular insulator. Once the conductors are introduced into the lumens they will kept well physically and electrically separated from each other.

A further advantage of the implantable medical lead of the embodiments with the conductors being movable in the lumens is that the conductors are easily accessible when electrically connecting the conductors to the electrodes and the electrode terminals of the implantable medical lead. In the prior art, the conductors must first be uncovered from the tight surrounding insulating material before they can be connected to any electrodes and electrode terminals. Thus, the surrounding insulating material is cut away from the conductor ends. There is then a risk that the very thin conductors can be damaged during this process step in the prior art. According to the embodiments, there is a space between the insulating walls of the lumens and the freely movable conductors. This means that if any insulating material of the tubular insulator needs to be removed, it can easily be cut away without the risk of damaging the conductors.

The embodiments described above are to be understood as a few illustrative examples of the present invention. It will be understood by those skilled in the art that various modifications, combinations and changes may be made to the embodiments without departing from the scope of the present invention. In particular, different part solutions in the different embodiments can be combined in other configurations, where technically possible. The scope of the present invention is, however, defined by the appended claims.

1. A method of manufacturing an implantable medical lead having a distal end and an opposite, proximal end configured to be mechanically and electrically connected to an implantable medical device, said method comprising:
extruding a polymer to form a tubular insulator having multiple separate lumens:
introducing a respective conductor in each lumen of said multiple separate lumens, wherein cross-sectional dimensions of said multiple separate lumens are defined relative cross-sectional dimensions of said respective conductor to enable movement of said respective conductor in a lumen of said multiple separate lumens relative said tubular insulator;
coiling said tubular insulator prior or after introducing said respective conductor to form a conductor coil having a coiled tubular insulator;
introducing said conductor coil into a bore of an insulating tubing of a lead body; and
electrically connecting each conductor of said conductor coil with an electrode of multiple electrodes arranged in connection with said distal end and an electrode terminal of multiple electrode terminals arranged in connection with said opposite, proximal end.

2. The method according to claim 1, wherein coiling said tubular insulator comprises coiling said tubular insulator prior or after introducing said respective conductor during heat treatment to form said conductor coil.

3. The method according to claim 1 wherein coiling said tubular insulator comprises coiling said tubular insulator after introducing said respective conductor to form said conductor coil.

4. The method according to claim 1, wherein extruding said polymer comprises extruding a polymer selected from the group consisting of polyurethane, a co-polymer of polyurethane and silicone, polyether ether ketone, ultra high molecular weight polyethylene, polyether block amide, polyamide and polyimide.

5. A method of manufacturing an implantable medical lead having a distal end and an opposite, proximal end configured to be mechanically and electrically connected to an implantable medical device, said method comprising:
providing an insulating core structure having a cross-shaped cross section to define four open channels;
introducing said insulating core structure in a bore of a first insulating tubing to form a tubular insulator having four separate lumens;
arranging a respective conductor in each of said four open channels prior to introducing said insulating core structure in said bore of said first insulating tubing or arranging said respective conductor in each of said four lumens after introducing said insulating core structure in said bore of said first insulating tubing, wherein each lumen of said four separate lumens houses a conductor and cross-sectional dimensions of said four separate lumens are defined relative cross-sectional dimensions of said conductors to enable movement of said conductors in said four separate lumens relative said tubular insulator;
coiling said tubular insulator to form a conductor coil having a coiled tubular insulator;
introducing said conductor coil into a bore of a second insulating tubing of a lead body; and
electrically connecting each conductor of said conductor coil with an electrode of multiple electrodes arranged in connection with said distal end and an electrode terminal of multiple electrode terminals arranged in connection with said opposite, proximal end.

6. The method according to claim 5, wherein
providing said insulating core structure comprises providing said insulating core structure made of a co-polymer of polyurethane and silicone, silicone, polyethylene, polybuthene, polypropylene or thermoplastic polyurethane having said cross-shaped cross section; and
introducing said insulating core structure comprises introducing said insulating core structure with said conductors in said bore of said first insulating tubing made of an elastic material selected from the group consisting of silicone and a co-polymer of polyurethane and silicone.

7. An implantable medical lead comprising:
multiple electrodes arranged in connection with a distal end of said implantable medical lead;
multiple electrode terminals arranged in connection with an opposite, proximal end of said implantable medical lead, wherein said opposite, proximal end is configured to be mechanically and electrically connected to an implantable medical device;
a lead body comprising an insulating tubing having a bore, wherein said lead body runs from said proximal end to said distal end; and
a conductor coil comprising a coiled tubular insulator having multiple separate lumens, wherein said conductor coil is arranged in said bore, each lumen of said multiple separate lumens houses a conductor and each conductor of said conductor coil is electrically connected to an electrode of said multiple electrodes and an electrode terminal of said multiple electrode terminals, wherein cross-sectional dimensions of said multiple separate lumens are defined relative cross-sectional dimensions of said conductors to enable movement of each conductor of said conductor coil in a lumen of said multiple separate lumens relative said coiled tubular insulator.

8. The implantable medical lead according to claim 7, wherein a largest outer cross-sectional dimension of said conductors is smaller than a smallest inner cross-sectional dimension of said multiple separate lumens.

9. The implantable medical lead according to claim 7, wherein each conductor of said conductor coil is electrically connected to a respective electrode of said multiple electrodes and a respective electrode terminal of said multiple electrode terminals.

10. The implantable medical lead according to claim 7, wherein said implantable medical lead comprises four electrodes arranged in connection with said distal end and four electrode terminals arranged in connection with said proximal end and said coiled tubular insulator comprises four separate lumens which are arranged so as to form a cross-sectional structure with a respective lumen in each of four quadrants.

11. The implantable medical lead according to claim 10, wherein two of said four conductors of said conductor coil are electrically connected to a first electrode of said multiple electrodes and a first electrode terminal of said multiple electrode terminals and the remaining two of said four conductors of said conductor coil are electrically connected to a second electrode of said multiple electrodes and a second electrode terminal of said multiple electrode terminals.

12. The implantable medical lead according to claim 7, wherein said multiple conductors are selected among wires, cables and coils of electrically conductive material.

13. The implantable medical lead according to claim 12, wherein said multiple conductors are coiled wires.

14. The implantable medical lead according to claim 7, wherein said multiple conductors have a diameter of no more than 0.1 mm, preferably of no more than 0.05 mm.

15. The implantable medical lead according to claim 7, wherein said multiple separate lumens comprises multiple pairs of co-radial lumens to form a co-radial, coaxial conductor coil.
